(19) United States
(12) Patent Application Publication
(10) Pub. No.: US 2010/0010532 A1
(43) Pub. Date: Jan. 14, 2010

(54) Device For Use In Treatment of Vericose Veins

(76) Inventor: Srinivasa Rao Vallabhaneni, Wirral (GB)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/922,293

(22) PCT Filed: Jun. 14, 2006
(86) PCT No.: PCT/GB2006/002174

§ 371 Date: Nov. 17, 2008

(30) Foreign Application Priority Data
Jun. 14, 2005 (GB) 0512073.8

(51) Int. Cl. A61M 29/00

(52) U.S. Cl. 606/198

(57) ABSTRACT

A device (10) for use in treatment of a varicose vein by deployment of the device therein, the device comprises a stem (15) carrying an openable occlusion component (12, 14) and means (22, 28) for anchoring the occlusion component within the vein.

This invention concerns a device for use in treatment of varicose veins.

Varicose veins develop as a consequence of abnormal blood flow within veins, commonly as a consequence of dysfunction of the valves of the veins. Treatment of this condition involves removal of veins from the circulation system and can be carried out by different methods.

Surgical treatment often involves ligation of the vein flush with the most proximal dysfunctional venous valve and physical removal of portions of the vein (in varying lengths). This operation requires surgical incision/s usually under general anaesthetic. Surgical incisions create a risk of bleeding, infection and formation of a scar. General anaesthetic carries a small risk of serious complications and can be associated with patient anxiety and some unpleasantness during recovery.

Transferring heat energy to the wall of the vein by different methods (e.g. radiofrequency electricity or laser) can be used to cause local effects within the vein wall leading to obliteration of the varicose vein. Such methods eliminate some or most of the surgical incisions. Injection of sclerosing agents into the veins is also an established treatment method. These substances cause irritation of the inner most layer of the vein leading to gradual fibrosis and obliteration of the lumen. This method is not widely used as the sole treatment for long or short saphenous system varicose veins. The method, however, is useful for treating isolated varicosities and to treat ‘spider veins’.

An object of this invention is to provide a device for use in treatment for varicose veins.

According to the invention there is provided a device for use in treatment of a varicose vein by deployment of the device therein, the device comprising a stem carrying an openable occlusion component and means for anchoring the occlusion component within the vein.

The occlusion component is preferably in the form of an umbrella, which is preferably made of fabric. The fabric used is ideally one that is or will in a relatively short time become impervious to blood.

The umbrella preferably has a skeletal frame preferably formed of radial spokes that are preferably linked by spring-like components for opening the umbrella when unrestrained. The spring-like components are preferably V-shaped connections between adjacent radial spokes.

In another preferred embodiment of the invention, the stem of the device comprises spring connecting radial spokes. The spring means may comprise springs connecting pairs of spokes for supporting the umbrella, which springs are preferably intercalated.

A preferred device of the invention has a guide means for a guide wire for deployment of the device. The guide means may be a guide hole through the stem longitudinally of the device. In the case of the stem comprising spring means, the guide hole may be through the spring means and especially through intercalated springs.

The anchoring means preferably comprise one or more tines that preferably extend from the stem of the device, so that the device can be pushed into a vein and then pulled back to engage the tines in valve leaflets or vein walls. The tines are preferably in pairs on opposite sides of the device and preferably there are two pairs of tines. The tines preferably extend from a head of the stem and preferably have barbed ends, in order to aid their retention in the valve leaflets or vein walls.

Additionally or alternatively the ends of the spokes of the skeletal frame may extend beyond the umbrella to provide the anchoring means.

Preferred devices according to the invention have one or more tails. The tails preferably extend from the occlusion component such as from the umbrella or part of the skeletal frame. A tail ideally comprises a plurality of threads and the threads are preferably linked by compressible lateral spacers, such as of shape memory elements. The spacers are preferably diamond shaped when unrestrained and preferably have a thread at each corner thereof.

The tails are preferably impregnated with a sclerosant, so that the inner most layer of the vein will be damaged resulting in scarring that will close the vein. The tails may be coated with sealing material, such as hydrogel.

Devices of the invention may also incorporate means for promoting sealing of the device in a vein, such as a hydrogel.

For deployment of the device of the invention, a pull string may be provided extending from the stem and a removable pushing member may be linked to the stem of the device. With these items, the device may be pushed into the vein past a preferred deployment position and then pulled back to engage the anchoring means in a desired deployment position. The pushing member may be linked to the stem by the or a pull string. The pushing member is preferably a hollow shaft and a portion of the pull string is contained within the shaft. Further, the device is preferably delivered into the vein in a deployment sheath surrounding at least the occlusion component in a constrained state and the sheath is then withdrawn to allow deployment of the device to take place. Deployment of the device may, for example, be guided by x-ray fluoroscopy or ultrasound scanning during the procedure.

The occluder devices of the invention may be used in conjunction with sclerotherapy where the device serves to prevent reflux of blood and cephalad flow of sclerosant material directly into the deep vein via the saphenofemoral or the saphenopopliteal junction. This may reduce the risk of sclerosant affecting the deep vein close to those junctions.

Hollow components of the or a delivery system for the devices of the invention may be used to deliver sclerosing agent, e.g. sclerosant foam, into a vein.

It is believed that the device of the invention will allow a mainly percutaneous and endoluminal method of treatment, eliminating or reducing surgical incisions. It may reduce or eliminate the need for general anaesthetic for the treatment of varicose veins and also lead to early discharge from hospital following treatment.

This invention will now further be described, by way of example only, with reference to the accompanying drawings, in which;

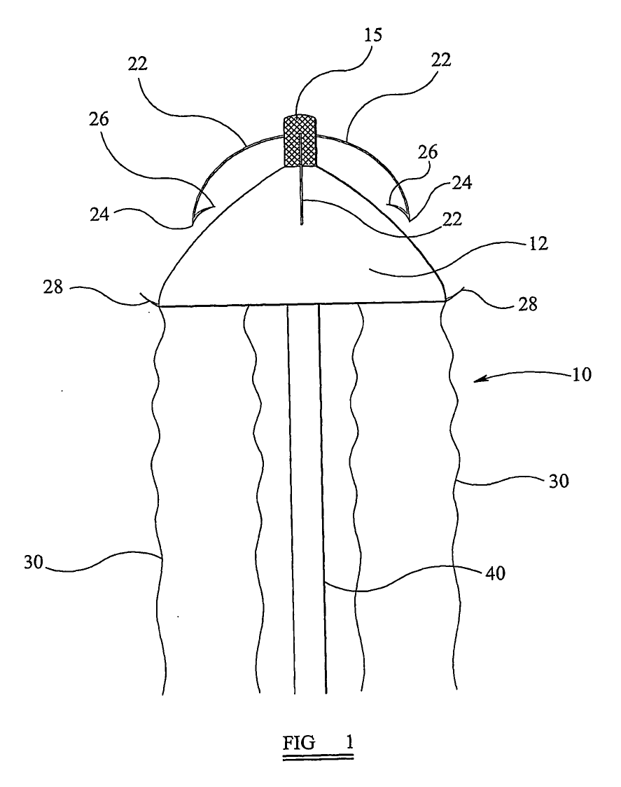

FIG. 1 shows an occlusion device according to the invention;

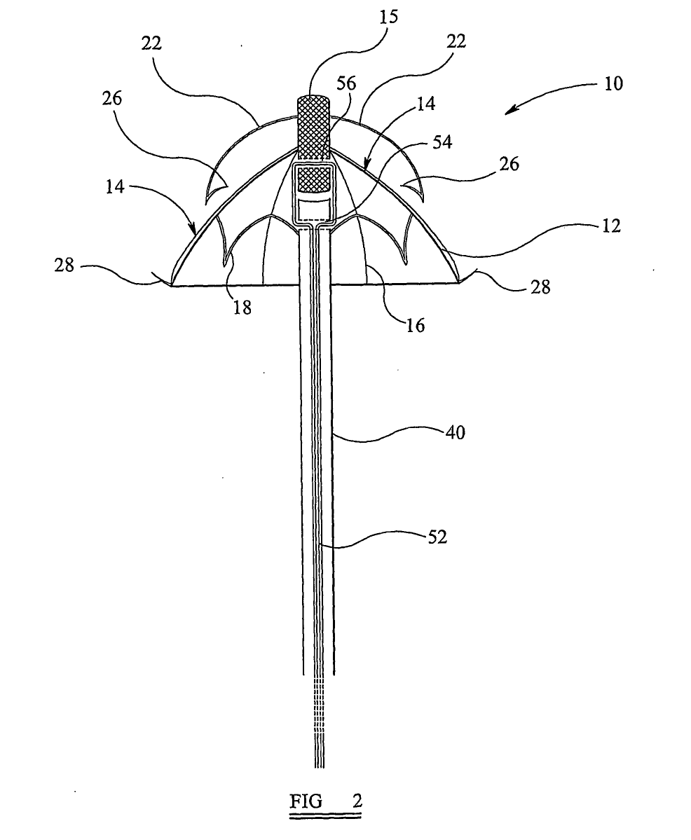

FIG. 2 is a longitudinal section through the device of FIG. 1;

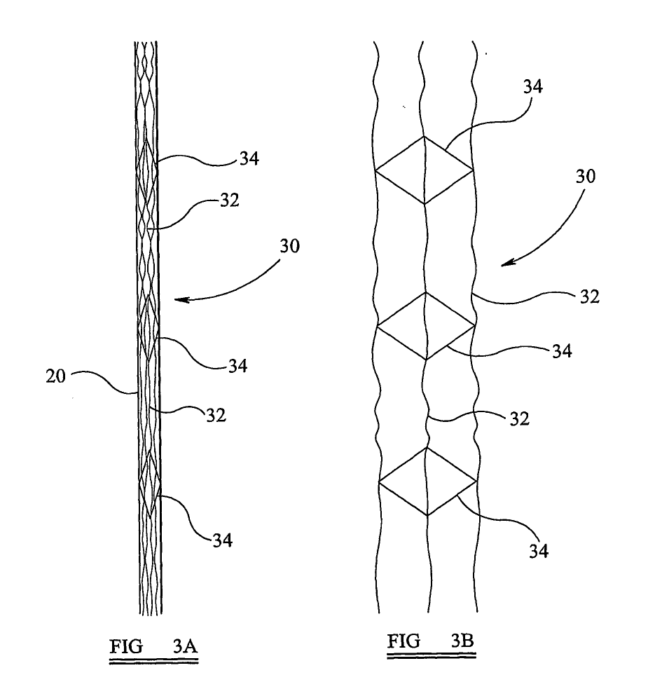

FIGS. 3A and 3B show a tail of the occlusion device of FIGS. 1 and 2 before and after deployment respectively;

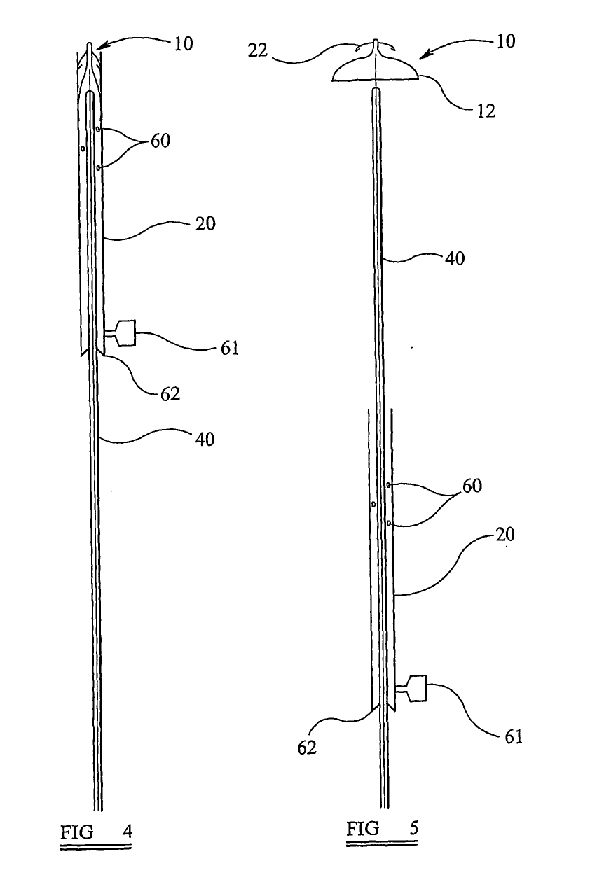

FIGS. 4 and 5 show deployment of the occlusion device of FIGS. 1 and 2;

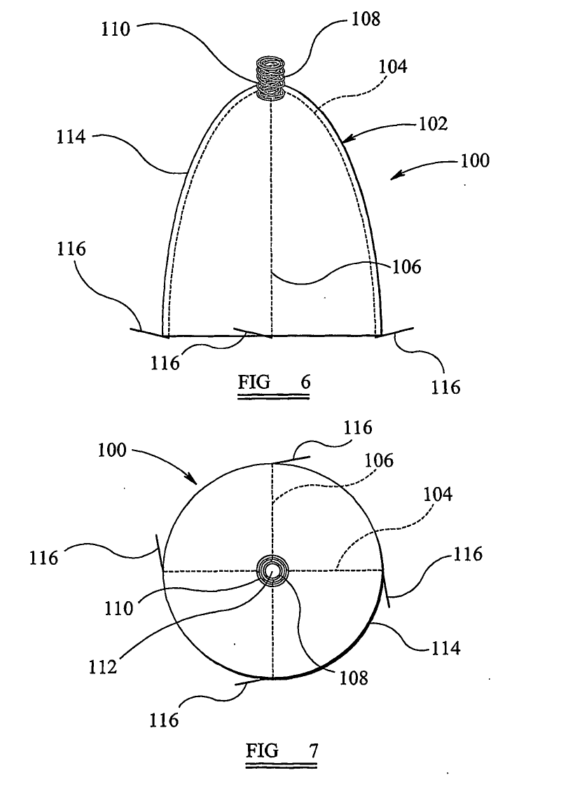

FIG. 6 is a side view of a second occlusion device according to the invention; and

FIG. 7 is a plan view of the device of FIG. 6.

Referring to FIGS. 1 to 5 of the accompanying drawings, as occlusion device 10 is made of a combination of a fabric umbrella 12 and a metal skeleton 14 on a stem 15. The occluder 10 is introduced into the lumen of the vein in a restrained state (i.e. with the umbrella closed) and opened-out at the target site so that it forms a barrier for blood flow through the vein.

The metal skeleton 14 comprises radial spokes 16 that are interconnected by “V” shaped metal wires 18. The connector wires 18 act as springs to provide radial force to open the umbrella 12 out and keep it in an opened state after deployment. The entire skeleton 14 is, however, flexible enough to allow packing into a narrow tube (sheath or catheter) 20 (see FIGS. 4 and 5) in a “closed umbrella” configuration to reduce its profile and facilitate endoluminal insertion. The umbrella fabric may be any that soon becomes impervious to blood. Multiple types of fibres may be used in the manufacture of the umbrella with properties that induce local thrombosis and robust incorporation. The fabric umbrella may be bonded to the skeleton by means of sutures. It may alternatively be possible to incorporate the metal skeleton into the warp and weave of the fabric to eliminate sutures.

The occluder 10 also has anchors 22 to hold the occluder in place and prevent migration. The anchors are multiple downwardly projecting tines relative to the closed end of the umbrella. Each tine has a sharp end 24 to engage down into a valve cusp and a barb 26 to prevent disengagement of an engaged tine. The anchors 22 are deployed just cephalad to the selected venous valve and the occluder is moved pulled) caudad so that the tines may engage (penetrate through) the valve leaflets of the vein walls. When the device 10 is unrestrained on a bench-top, the tines would have a lower profile than the umbrella. The aim is to engage the valve leaflets with the tines, so the profile should be sufficiently large to engage but not too large to hit the vein wall. However, should the tines engage the wall instead of the valve leaflets, there is scope for little harm. The tines 22 are also shorter than the spokes 16 of the occulder. This discrepancy in the length is necessary to divide deployment into stages. The spokes 16 of the umbrella 12 may be equipped with short and sharp upturned ends 28 that may enhance fixation.

The umbrella occluder 10 had tails 30 that extend downwardly relative to the open end of the umbrella. The tails 30 are made of threads 32 that are attached to the umbrella. The threads may be attached to the skeleton of the occluder in a manner similar to that of attaching threads to atruamatic suture needles or by other means. It is also possible for the threads used to weave the fabric of the umbrella to be left long. These tails 30 are left trailing in the lumen of the vein up to the point of insertion of the device and incorporated into a ligature that goes round the vein. The tails may also be impregnated with a sclerosant, elution of which expedites and ensures obliteration of the vein. The tails 30 act as further safeguard against migration of the device 10.

The tail threads 32 have four-sided spacers 34 at intervals (see FIGS. 3a and 3b). The spacers 34 open into diamond shapes upon deployment, thus flattening the vein lumen and improving the contact between the wall and the tail threads. The spacers may be of any suitable closed loop form. A function of the tails is to introduce sclerosant eluting medium and to provide additional (temporary or permanent) fixation (and not primarily occlusion of the lumen).

For deployment of the occluder 10, it is detachably attached to a shaft 40, which may be of metal or other suitable material. The device is then folded and together with the shaft packed into the catheter-based system (sheath) 20 to reduce its profile. The occluder is deployed in stages by incrementally withdrawing the sheath. The sheath 20 may have one or more holes 60 distally to allow flow of contrast material for imaging and an attachment 61 at the lower end to attach a contrast filled syringe.

Ideally, the occluder is amendable to both push and pull movements to achieve the different deployment steps, viz. engage the anchor and to position and deploy the occluder. One possible design of deployment device is shown in FIGS. 4 and 5 of the drawings.

The umbrella occluder 12 needs stability and manoeuvrability to allow positioning at the target location and to pull downwards to allow the anchor to engage the valve leaflets. The central shaft 40 provides “pushability”. The attachment between the shaft 40 and the occluder 10 provides “pullability”. This attachment should also be readily detachable when required. To acheive this, there are pull strings 52 extending through the lumen in the hollow shaft, coming out through holes 54 and going through an eye 56 in the stem 15 of the umbrella 12. The pull strings 52 traverse all the way through the shaft 40 and at the lower end of the shaft are attached in a way that can be undone easily. When intending to remove the shaft, this attachment is undone and the shaft removed, leaving the occluder in its place. The pull strings are trimmed along with the tail and act thereafter as part of the tail. Alternatively, the pull string may be removed.

To deploy the occluder in practice, a patient is placed supine on an operating table. The sheathed device 10 is introduced into the target vein distally via a venotomy made under local anaesthetic. The device is manoeuvred cephalad through the lumen of the vein and once it is approximately in the appropriate position, imaging is done to confirm relationship with the target valve. Contrast material for imaging may be injected through the sheath 20 or via a separate catheter. A valve 62 may be provided to prevent the contrast material flowing out at the lower end of the sheath 20.

The patient is tilted head-up or head-down depending upon the stage of the procedure. The anchor tines are first deployed just above the valve leaflets and the sheathed device is pulled caudad by means of the pull strings 52 to engage the anchor tines. Then the occluder is deployed by withdrawing the sheath. The shaft, together with the “pull strings” provide the stability for the occluder when the sheath is being withdrawn. The spacers may cause friction between the tail and the sheath and without this stability, the device may be pulled down. Once the unsheathing is completed and there is minimal need to maintain push/pull stability on the device, the pull strings are disengaged from the shaft. The shaft is then removed. Maintaining X-ray visibility of the occluder during this stage reassures the operator regarding the fixation. Once the delivery system components are completely removed from the body, the pull strings and the tail are together trimmed to about the level of the venotomy and are incorporated into a ligature that surrounds the vein. Wounds are closed in the standard manner.

Turning to FIGS. 6 and 7 of the accompanying drawings, there is shown another occluder device 100 according to the invention. The device 100 has a framework 102 consisting of four radial spokes in pairs 104, 106. Each spoke of a pair of spokes extends from an opposite end of a coil spring 108, 110. The coil springs 108, 110 are intercalated and provide a guide hole 112, through which a guide wire can be fed for deployment of the device. The framework 102 is covered by a fabric umbrella 112. The fabric umbrella may be bonded to the framework by means of sutures or the framework may be incorporated into the warp and weave of the fabric to avoid use of sutures.

The spokes of the framework have out-turned ends 116 for fixation of the device in a vein by hooking into the vein wall.

As in the first illustrated embodiment the device 100 may be deployed in a vein by enclosing the device in a sheath and delivering the sheathed device over a guide wire. Withdrawal of the sheath when the device is in a desired position will allow the spokes 104, 106 to spring outwards due to their being connected by coiled springs 108, 110 and the out-turned ends 116 to engage the vein wall.

The illustrated occlusion devices may incorporate sealing material such as a hydrogel to improve sealing of the umbrella against a vein wall.

The device 100 is used in a similar manner to the device 10 of FIGS. 1 to 5.

1. A device for use in treatment of a varicose vein by deployment of the device therein, the device comprising a stem carrying an openable occlusion component and means for anchoring the occlusion component within the vein.

2. A device as claimed in claim 1, wherein the occlusion component comprises an umbrella.

3. A device as claimed in claim 2, wherein the umbrella is of fabric.

4. A device as claimed in claim 2, wherein the umbrella has a skeletal frame.

5. A device as claimed in claim 4, wherein the skeletal frame has spring-like components for opening the umbrella when unrestrained.

6. A device as claimed in claim 4, wherein the skeletal frame comprises radial spokes.

7. A device as claimed in claim 5, wherein the spring-like components are V-shaped connections between adjacent radial spokes.

8. A device as claimed in claim 6, wherein the stem comprises spring means connecting radial spokes.

9. A device as claimed in claim 8, wherein the spring means comprise springs connecting pairs of spokes.

10. A device as claimed in claim 9, wherein said springs are intercalated.

11. A device as claimed in claim 1, having guide means for a guide wire for deployment of the device,

12. A device as claimed in claim 11, wherein the guide means is a guide hole through the stem longitudinally of the device.

13. A device as claimed in claim 11, wherein the stem comprises spring means connecting radial spokes, and the guide means is a guide hole through the spring means.

14. A device as claimed in claim 6, wherein the anchoring means comprises out-turned ends of the radial spokes.

15. A device as claimed in claim 1, wherein the anchoring means comprise one or more tines.

16. A device as claimed in claim 15, wherein the tines extend from a head of the stem.

17. A device as claimed in claim 16, wherein the tines have barbed ends.

18. A device as claimed in claim 1, having one or more tails.

19. A device as claimed in claim 18, wherein the tails extend from the occlusion component.

20. A device as claimed in claim 18, wherein the tails comprises a plurality of threads.

21. A device as claimed in claim 20, wherein the threads are linked by compressible lateral spacers.

22. A device as claimed in claim 21, wherein the spacers are shape memory elements.

23. A device as claimed in claim 21, wherein the spacers are closed loops when unrestrained.

24. A device as claimed in claim 18, wherein the tails are impregnated with the sclerosant.

25. A device as claimed in claim 1, further comprising a pull string extending from the stem

26. A device as claimed in claim 1, further comprising a removable pushing member inked to the stem.

27. A device as claimed in claim 26, wherein the pushing member is linked to the stem by the or a pull string.

28. A device as claimed in claim 27, wherein the pushing member is a hollow shaft and a portion of the pull string is contained within the shaft.

29. A device as claimed in claim 1, further comprising a deployment sheath surrounding at least the occlusion component in a constrained state.

30. A device as claimed in claim 1, incorporating means for promoting sealing of the device in a vein.

31. A device as claimed in claim 29, wherein the sealing means is a hydrogel.

32. A device as claimed in claim 30, having one or more tails, wherein the sealing means coats the tails.

33. (canceled)
